(19) United States
(12) Patent Application Publication
(10) Pub. No.: US 2018/0064344 A1
(43) Pub. Date: Mar. 8, 2018

(54) Tremor Reduction Device

(71) Applicant: California State University Fresno Foundation, Fresno, CA (US)

(72) Inventor: The Nguyen, Fresno, CA (US)

(21) Appl. No.: 15/601,201

(22) Filed: May 22, 2017

Related U.S. Application Data
(60) Provisional application No. 62/383,287, filed on Sep. 2, 2016.

(51) Int. Cl. A61B 5/00; F16F 15/02; F16F 15/03; A63B 21/22

(52) U.S. Cl. CPC A61B 5/0053; F16F 15/022; F16F 15/03; A61B 5/1101; A63B 21/22; A61B 5/4076; A61B 5/6824

(57) ABSTRACT

A wearable tremor reduction device reduces tremor by internally generating forces which cancel or reduce the magnitude force of the tremor experienced by the person wearing the device. The device may be worn on a wrist, arm, ankle or leg. The device has a plurality of housing members which are flexibly connected together. Each housing member contains a mass which is translatable along an axis between a proximal limit and a distal limit, and a neutral position midway between the proximal limit and the distal limit. Following imposition of a force having a component along the axis, a biasing means returns the mass to the neutral position.

### RELATED APPLICATIONS

This application claims domestic priority to U.S. Provisional Application 62/383,287 filed Sep. 2, 2016.

### BACKGROUND OF THE INVENTION

Tremors of various extremities of the human body are common movement disorders, characterized by rhythmic oscillations of the extremity around one or more joints. One of the most important characteristics to be assessed in a patient's tremor is the tremor frequency, which is measured in oscillations per second or cycles per second (Hz). Another important characteristic of tremor is amplitude, which is the linear or angular displacement of the limb or body part. Tremor amplitude is measured in millimeters or degrees. Tremor frequency and amplitude can be measured to a relatively high degree of accuracy by known apparatus and methods.

Tremors which are caused by Parkinson's disease, i.e., Parkinsonian tremor, and essential tremor can significantly impact the quality of life of the person suffering from these maladies. Daily functions, such as eating, combing one's hair, brushing teeth, etc.,—which functions are generally taken for granted by persons without tremors—can be demanding and frustrating exercises for those suffering from tremors. Tremor magnitude is frequently reported as being the greater problem from persons suffering from the disorder, because it has a greater impact on a person's ability to perform these daily activities. Because of these issues, a variety of solutions to tremors have been proposed to provide relief from persons suffering from tremor.

While drug therapy has been employed to provide relief for persons suffering from these tremors, the expense and potential side effects of the treatment can be an obstacle. There have also been efforts to provide relief without using drugs utilizing elector-mechanical or mechanical devices. For example, one device utilizes a wearable tremor suppression exoskeleton, which appears as a robotic external structure. In the absence of external forces, this device applies dynamic internal forces on the upper limb programmed to reduce the tremor by applying biomechanical loads. Another device is a wearable orthosis which uses a DC motor to reduce tremors at each joint. Some tremor suppression devices attempt to suppress the tremors by application of pressure at a specific location on a human extremity to induce a tremor suppressing stimulus. Another device uses a forearm, wrist and splint to attach at least one gyroscope. Other devices utilize braces having compartments containing a viscous fluid to dampen wrist flexion and extension tremor. Another device is a weighted glove which is customizable by size. Patients are able to adjust the weight of the glove by filling integrated pockets in the glove with circular weight disks.

With many of the known devices, the persons wearing the devices have complained that the devices are bulky, uncomfortable, unsightly, too heavy, or simply did not provide a satisfactory solution to their tremor problem.

Thus, there is a need for a simple, relatively light, inconspicuous and effective device which suppresses tremors caused by Parkinson's disease and essential tremor.

### SUMMARY OF THE INVENTION

Embodiments of the present invention provide a solution to the need identified above. Embodiments of the invention create neutralizing forces which cancel or reduce the magnitude of the tremor experienced by the person wearing the device. Parkinsonian tremor typically has a frequency in the range of 3-7 Hz, while essential tremor typically has a frequency in the range of 4-12 Hz. The tremors may have a vertical component, a horizontal component, and a rotational component.

Embodiments of the invention comprise a plurality of housing members flexibly connected together. Each housing member contains a mass which is translatable along an axis between a proximal limit and a distal limit, and a neutral position midway between the proximal limit and the distal limit, where, following imposition of a force having a component along the axis, a biasing means returns the mass to the neutral position.

The plurality of housing members in an embodiment of the invention may comprise a first housing member, a second housing member, a third housing member and a fourth housing member. The first housing member may have a first proximal sidewall and a first distal sidewall, where the first proximal sidewall and the first distal sidewall are in opposite facing spaced-apart relation. A first mass is disposed between the first proximal side wall and the first distal side wall, where the first mass may have a first proximal side and a first distal side. A first proximal spring is disposed between the first proximal side wall and the first proximal side of the first mass, and a first distal spring is disposed between the first distal side wall and the first distal side of the first mass.

Likewise, the second housing member may have a second proximal sidewall and a second distal sidewall in opposite facing spaced-apart relation. A second mass is disposed between the second proximal side wall and the second distal side wall, where the second mass has a second proximal side and a second distal side. A second proximal spring disposed between the second proximal side wall and the second proximal side of the second mass, and a second distal spring is disposed between the second distal side wall and the second distal side of the second mass.

In similar fashion, the third housing member may have a third proximal sidewall and a third distal sidewall in opposite facing spaced-apart relation. A third mass is disposed between the third proximal side wall and the third distal side wall, where the third mass has a third proximal side and a third distal side. A third proximal spring is disposed between the third proximal side wall and the third proximal side of the third mass, and a third distal spring disposed between the third distal side wall and the third distal side of the third mass.

The linked housing members are completed by a fourth housing member which may have a fourth proximal sidewall and a fourth distal sidewall in opposite facing spaced-apart relation. A fourth mass is disposed between the fourth proximal side wall and the fourth distal side wall, where the fourth mass has a fourth proximal side and a fourth distal side. A fourth proximal spring is disposed between the fourth proximal side wall and the fourth proximal side of the fourth mass, and a fourth distal spring disposed between the fourth distal side wall and the fourth distal side of the fourth mass.

In the embodiment described above, the first housing member is flexibly linked to the second housing member, the second housing member is flexibly linked to the third housing member, the third housing member is flexibly linked to the fourth housing member, and the fourth housing member is flexibly linked to the first housing member thereby forming an interconnected band adapted to encircle a limb of the user.

Also disclosed herein is a method for reducing tremors in a limb of a person caused by Parkinson's disease or essential tremor utilizing the following steps. First the horizontal and vertical frequency of the tremors is measured on the person suffering from the condition. Once this information is determined for a particular person suffering from Parkinson's disease or essential tremor, a tremor reduction apparatus is configured, where the tremor reduction apparatus has a plurality of housings linked together in a closed loop configuration. Each of the plurality of housings contains its own mass which is translatable along an axis between a proximal limit and a distal limit set within the housing. A neutral position is defined between the proximal limit and the distal limit. Each of the housings also contains a biasing means for returning the mass to the neutral position. The mass and biasing means are sized according to the horizontal frequency and the vertical frequency of the measure tremors. The configured tremor reduction apparatus is then placed about the limb of the person.

Embodiments of the present invention may be designed to address the tremors suffered by a particular patient. The frequency and amplitude of a person's tremors may be measured on each extremity. Once the frequency and amplitude are known, the size of the mass contained within each of the housing members may be specified. Alternatively, or in addition, the magnitude of the biasing means may be adjusted. For example, if springs are utilized as a biasing means, the spring force may be adjusted by either using springs having a different spring force, or increasing or decreasing the number of springs. The installation of different mass sizes and/or different biasing means is facilitated with embodiments of the present invention which have housings which are adapted to receive a variety of interchangeable weights and springs which may be exchanged within the housing with relative ease.

The housing members of embodiments of the invention may be linked together with straps, belts, netting, or other flexible and light connecting fabric which is suitable for linking the housings and securing the housings about a patient's arm or leg. Embodiments of the invention may further comprise a memory foam layer between the device itself and patient's arm or leg so that the device fits snugly tight, but is not uncomfortable.

In modeling tremor and the effectiveness of embodiments of the present invention, the inventor herein found that by utilizing the present invention, the magnitude of tremor is substantially reduced. For example, utilizing a machine which models tremor having a frequency of 4 to 5 Hz, without the device the tremor had a horizontal magnitude of 10 millimeters and a vertical magnitude of 21 millimeters. With an embodiment of the invention placed about the “wrist” utilized in the model, the horizontal magnitude was reduced to 3.5 millimeters and the vertical magnitude was reduced to 7.0 millimeters.

### BRIEF DESCRIPTION OF THE DRAWINGS

FIG. 1 shows a front perspective view of an embodiment of the disclosed tremor reduction device placed about the wrist of a user.

FIG. 2 shows a front perspective view of the embodiment depicted in FIG. 1 an embodiment of the invention secured about the wrist of a user with a strap and hook and loop fasteners, with housing covers in place

FIG. 3 shows a top perspective view of an alternative embodiment.

FIG. 4 shows a front side perspective view of an embodiment utilizing a exterior strap for securing to the wrist of a user.

FIG. 5 shows a side perspective view of the embodiment depicted in FIG. 4.

FIG. 6 shows a front side perspective view of the embodiment depicted in FIG. 4 with the housing covers removed.

FIG. 7 shows a side perspective view of the embodiment depicted in FIG. 4 with the housing covers removed.

FIG. 8 shows a front perspective view of the embodiment depicted in FIG. 4 with the housing covers removed.

### DETAILED DESCRIPTION OF THE INVENTION

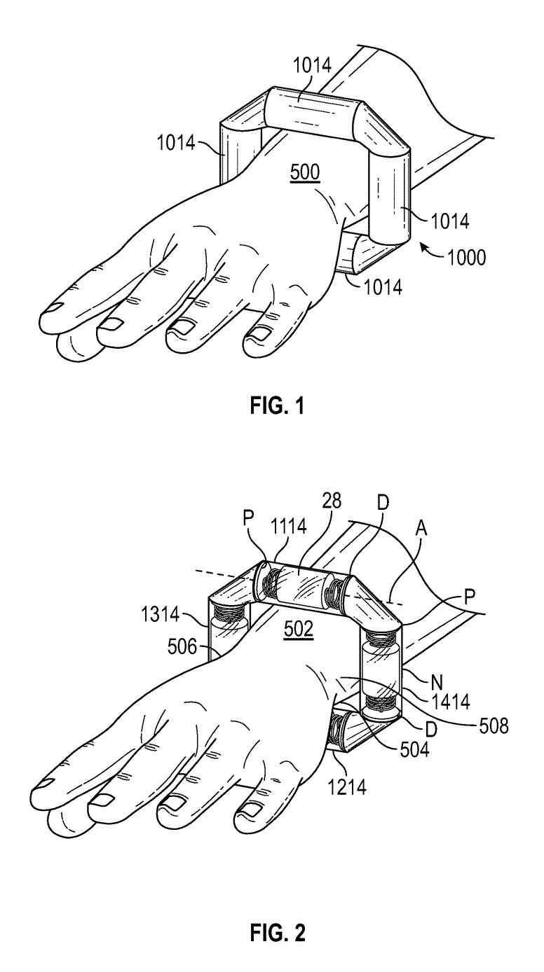

Referring now to the figures, FIG. 1 shows a first embodiment of the disclosed tremor reduction device 1000, as worn around a user's wrist 500. In one embodiment of the invention, the tremor reduction device 1000 has a plurality of housing members 1014 flexibly connected together. The embodiment shown in FIG. 1 has four housing members 1014 flexibly connected together to encircle the user's wrist 500, with a first housing member 1114 adjacent to the top side 502 of the wrist, a second housing member 1214 adjacent to the palm side 504 of the wrist, a third housing member 1314 adjacent the thumb side 506 of the wrist, and a fourth housing member 1414 adjacent to the the little finger side 508 of the wrist 500. Each of the housing members 14 contains a mass 28 which is translatable along an axis A between a proximal limit P and a distal limit D, and a neutral position N midway between the proximal limit P and the distal limit D, wherein, following imposition of a force having a component along the axis, a biasing means returns the mass to the neutral position.

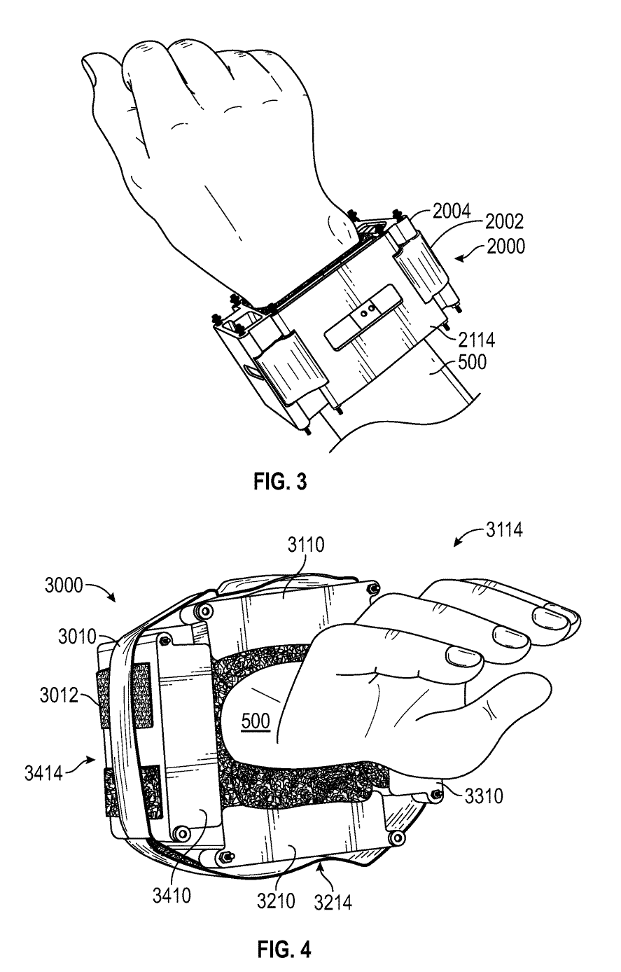

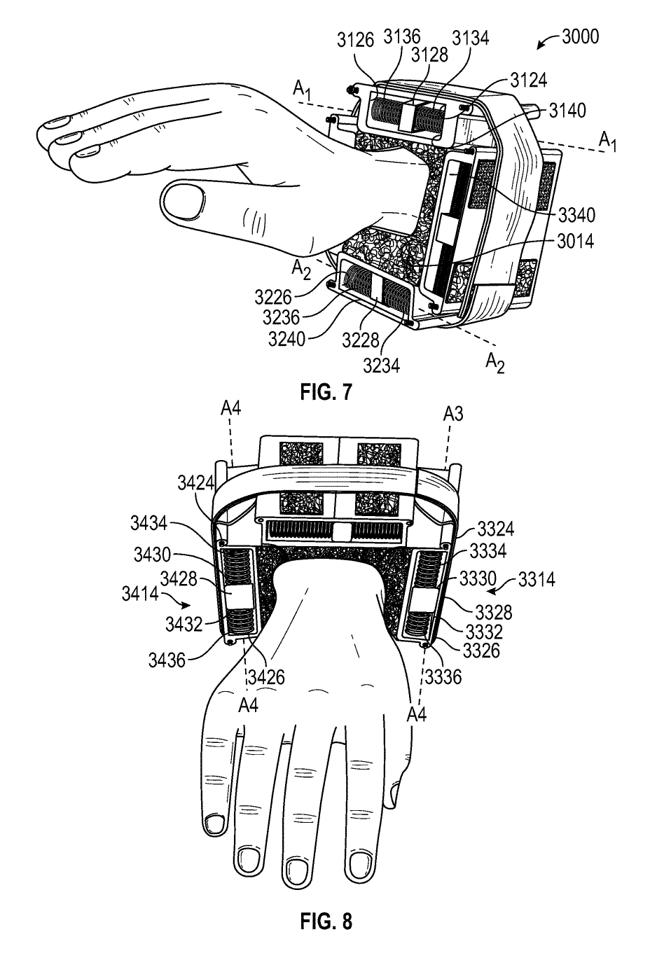

A second embodiment and a third embodiment of the tremor reduction apparatus are depicted in FIGS. 3 through 8. The second embodiment 2000 is secured about the user's wrist 500 by a strap 2002 which loops around strap posts of adjacent housing members 2114. Strap 2002 will have an attachment mechanism, such as a buckle, clasp, snap buttons, or hook and loop fasteners, for securing the strap and enclosing the apparatus around the user's wrist. Housing members 2114 of the second embodiment 2000 are identical to the housing members 3114 of the third embodiment 3000 and are described in detail below.

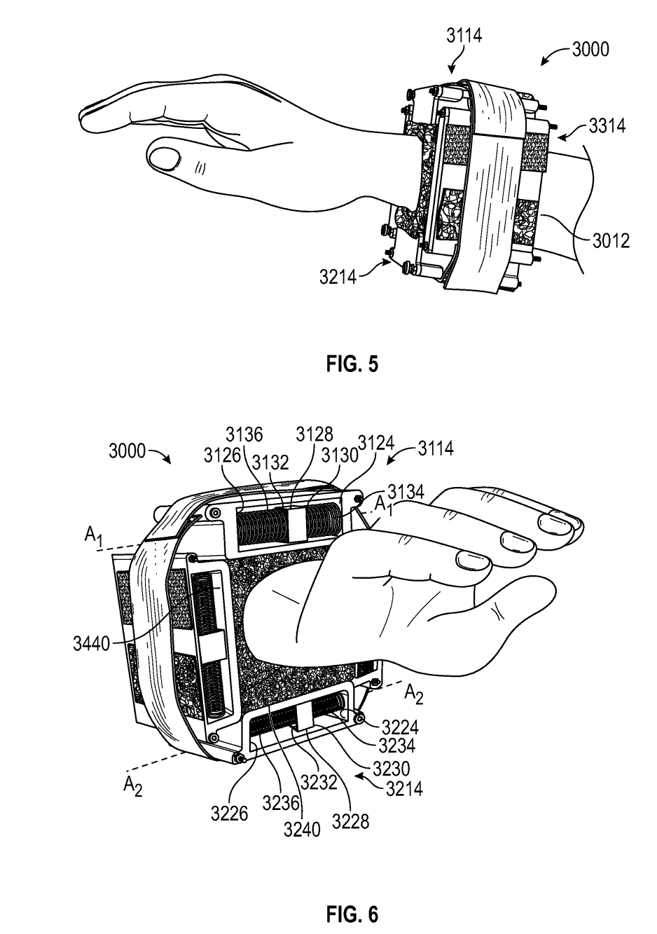

FIGS. 4-5 depict third embodiment 3000 of the tremor reduction device wherein first housing member 3114 has a housing cover 3110, second housing member 3214 has a housing cover 3210, third housing member 3314 has a housing cover 3310, and fourth housing member 3414 has a housing cover 3410. Third embodiment 3000 may be secured around the user's wrist 500 by a continuous strap 3010 which wraps around the outsides of the housing members 3114, 3214, 3314 and 3414. Each housing cover 3110, 3214, 3314 and 3414 may have attachment means, such as piece of hook and loop fastener, 3012, for further securing continuous strap 3010. The inside surface of the tremor reduction device may comprise a layer of memory foam 3014 which may be disposed between any embodiment 1000, 2000, 3000 of the tremor reduction device and the wrist 500 of the user.

FIGS. 4-8 depict a third embodiment 3000 of the tremor reduction device. As shown in FIGS. 4 through 8, the housing members of the tremor reduction device 3000 may have the following configuration. First housing member 3114 may have a first proximal sidewall 3124 and a first distal sidewall 3126, with the first proximal sidewall 3124 and the first distal sidewall 3126 in opposite facing spaced-apart relation. A first mass 3128 is disposed between the first proximal side wall 3124 and the first distal side wall 3126. The first mass 3128 has a first proximal side 3130 and a first distal side 3132. A first proximal spring 3134 is disposed between the first proximal side wall 3124 and the first proximal side 3130 of the first mass 3128. A first distal spring 3136 is disposed between the first distal side wall 3126 and the first distal side 3132 of the first mass 3128. First mass 3128 is translatable along axis A₁ between a first proximal limit set by first proximal side wall 3124 and first distal side 3132, where following imposition of a first force having a component along axis A₁, first proximal spring 3134 and first distal spring 3136 return first mass 3128 to a first neutral position at an approximate midpoint between first proximal side wall 3124 and first distal side wall 3126. FIGS. 6-8 depict first mass 3128 at the first neutral position. It is to be appreciated that during a tremor episode the first mass 3128 may be in near constant oscillation between the first proximal side wall 3124 and the first distal side wall 3126, such that the first mass 3128 does not come to a static condition at the first neutral position. The first neutral position is achieved when no external forces—such as a tremor—act upon the first mass.

First proximal sidewall 3124 and first distal sidewall 3126 may be attached to or may be integral to a first base plate 3140. First base plate 3140 may have a sliding means upon which the first mass 3128 may slidably translate. The sliding means may include a rail member, magnetic levitation, air suspension or a layer of a friction reducing medium, such as balls.

Likewise, second housing member 3214 may have a second proximal sidewall 3224 and a second distal sidewall 3226, with the second proximal sidewall 3224 and the second distal sidewall 3226 in opposite facing spaced-apart relation. A second mass 3228 is disposed between the second proximal side wall 3224 and the second distal side wall 3226. The second mass 3228 has a second proximal side 3230 and a second distal side 3232. A second proximal spring 3234 is disposed between the second proximal side wall 3224 and the second proximal side 3230 of the second mass 3228. A second distal spring 3236 is disposed between the second distal side wall 3226 and the second distal side 3232 of the second mass 3228. Second mass 3228 is translatable along axis A₂ between a second proximal limit set by second proximal side wall 3224 and second distal side 3232, where following imposition of a second force having a component along axis A₂, second proximal spring 3234 and second distal spring 3236 return second mass 3228 to a second neutral position at an approximate midpoint between second proximal side wall 3224 and second distal side wall 3226. FIGS. 6-8 depict second mass 3228 at the second neutral position. It is to be appreciated that during a tremor episode the second mass 3228 may be in near constant oscillation between the second proximal side wall 3224 and the second distal side wall 3226, such that the second mass 3228 does not come to a static condition at the second neutral position. The second neutral position is achieved when no external forces—such as a tremor—act upon the second mass.

Second proximal sidewall 3224 and second distal sidewall 3226 may be attached to or may be integral to a second base plate 3240. Second base plate 3240 may have a sliding means upon which the second mass 3228 may slidably translate. The sliding means may include a rail member, magnetic levitation, air suspension or a layer of a friction reducing medium, such as balls.

Likewise, third housing member 3314 may have a third proximal sidewall 3324 and a third distal sidewall 3326, with the third proximal sidewall 3324 and the third distal sidewall 3326 in opposite facing spaced-apart relation. A third mass 3328 is disposed between the third proximal side wall 3324 and the third distal side wall 3326. The third mass 3328 has a third proximal side 3330 and a third distal side 3332. A third proximal spring 3334 is disposed between the third proximal side wall 3324 and the third proximal side 3330 of the third mass 3328. A third distal spring 3336 is disposed between the third distal side wall 3326 and the third distal side 3332 of the third mass 3328. Third mass 3328 is translatable along axis A₃ between a third proximal limit set by third proximal side wall 3324 and third distal side 3332, where following imposition of a third force having a component along axis A₃, third proximal spring 3334 and third distal spring 3336 return third mass 3328 to a third neutral position at an approximate midpoint between third proximal side wall 3324 and third distal side wall 3326. FIGS. 6-8 depict third mass 3328 at the approximate third neutral position. It is to be appreciated that during a tremor episode the third mass 3328 may be in near constant oscillation between the third proximal side wall 3324 and the third distal side wall 3326, such that the third mass 3328 does not come to a static condition at the third neutral position. The third neutral position is achieved when no external forces—such as a tremor—act upon the third mass.

Third proximal sidewall 3324 and third distal sidewall 3326 may be attached to or may be integral to a third base plate 3340. Third base plate 3340 may have a sliding means upon which the third mass 3328 may slidably translate. The sliding means may include a rail member, magnetic levitation, air suspension or a layer of a friction reducing medium, such as balls.

Likewise, fourth housing member 3414 may have a fourth proximal sidewall 3424 and a fourth distal sidewall 3426, with the fourth proximal sidewall 3424 and the fourth distal sidewall 3426 in opposite facing spaced-apart relation. A fourth mass 3428 is disposed between the fourth proximal side wall 3424 and the fourth distal side wall 3426. The fourth mass 3428 has a fourth proximal side 3430 and a fourth distal side 3432. A fourth proximal spring 3434 is disposed between the fourth proximal side wall 3424 and the fourth proximal side 3430 of the fourth mass 3428. A fourth distal spring 3436 is disposed between the fourth distal side wall 3426 and the fourth distal side 3432 of the fourth mass 3428. Fourth mass 3428 is translatable along axis A₄ between a fourth proximal limit set by fourth proximal side wall 3424 and fourth distal side 3432, where following imposition of a fourth force having a component along axis A₄, fourth proximal spring 3434 and fourth distal spring 3436 return fourth mass 3428 to a fourth neutral position at an approximate midpoint between fourth proximal side wall 3424 and fourth distal side wall 3426. FIGS. 6-8 depict fourth mass 428 at the fourth neutral position. It is to be appreciated that during a tremor episode the fourth mass 3428 may be in near constant oscillation between the fourth proximal side wall 3424 and the fourth distal side wall 3426, such that the fourth mass 3428 does not come to a static condition at the fourth neutral position. The fourth neutral position is achieved when no external forces—such as a tremor—act upon the fourth mass.

Fourth proximal sidewall 3424 and fourth distal sidewall 3426 may be attached to or may be integral to a fourth base plate 3440. Fourth base plate 3440 may have a sliding means upon which the fourth mass 3428 may slidably translate. The sliding means may include a rail member, magnetic levitation, air suspension or a layer of a friction reducing medium, such as balls.

As shown in the Figures, the axes of translation of each side of the apparatus are in parallel alignment. That is, axis A₁ is in parallel alignment with axis A₂ and axis A₃ is in parallel alignment with axis A₄. In this configuration, the translation of the first mass 3128 along axis A₁ and the translation of the second mass 3228 along axis A₂ collectively reduce tremor along a first translational direction which is parallel to axis A₁ and axis A₂. Likewise, the translation of the third mass 3328 along the axis A₃ and the translation of the fourth mass 3428 along axis A₄ reduce tremor along a second translational direction which is parallel to axis A₃ and A₄. the sum of the forces generated by the translation of first mass 3128 along axis A₁, second mass 3228 along axis A₂, third mass 3328 along axis A₃ and fourth mass along axis A₄ reduce tremor having a rotational direction

The different embodiments 1000, 2000, 3000 may have interchangeable weight for use as the required mass (e.g., 3128, 3218, 3318, and 3418 for the third embodiment) for providing the required force for neutralizing the force generated by the tremor. Likewise, the spring size and stiffness can be adjusted as necessary to provide the required biasing force for translation of each of the masses. Embodiments of the apparatus may have features which facilitate interchanging different weights and springs to allow empty housings to be configured as required for the needs of a particular patient.

Also disclosed herein is a method for reducing tremors in a limb of a person caused by Parkinson's disease or essential tremor utilizing the following steps. First the horizontal and vertical frequency of the tremors is measured on the person suffering from the condition. Once this information is determined for a particular person suffering from Parkinson's disease or essential tremor, a tremor reduction apparatus is configured, such as one of the embodiments 1000, 2000, 3000 of the presently disclosed apparatus. As described above, the tremor reduction apparatus has a plurality of housings linked together in a closed loop configuration. Each of the plurality of housings contains its own mass which is translatable along an axis between a proximal limit and a distal limit set within the housing. A neutral position is defined between the proximal limit and the distal limit. Each of the housings also contains a biasing means for returning the mass to the neutral position. The mass and biasing means are sized according to the horizontal frequency and the vertical frequency of the measure tremors. The configured tremor reduction apparatus is then placed about the limb of the person.

Having thus described the preferred embodiment of the invention, what is claimed as new and desired to be protected by Letters Patent includes the following:

What is claimed is:

1. A tremor reduction device worn by a user, the device comprising:
a plurality of housing members flexibly connected together, each housing member containing a mass which is translatable along an axis between a proximal limit and a distal limit, and a neutral position midway between the proximal limit and the distal limit, wherein, following imposition of a force having a component along the axis, a biasing means returns the mass to the neutral position.

2. The tremor reduction device of claim 1 wherein the plurality of housing members comprise:
a first housing member comprising a first proximal sidewall and a first distal sidewall, the first proximal sidewall and the first distal sidewall in opposite facing spaced-apart relation, a first mass disposed between the first proximal side wall and the first distal side wall, the first mass comprising a first proximal side and a first distal side, a first proximal spring disposed between the first proximal side wall and the first proximal side of the first mass, and a first distal spring disposed between the first distal side wall and the first distal side of the first mass;
a second housing comprising a second proximal sidewall and a second distal sidewall, the second proximal sidewall and the second distal sidewall in opposite facing spaced-apart relation, a second mass disposed between the second proximal side wall and the second distal side wall, the second mass comprising a second proximal side and a second distal side, a second proximal spring disposed between the second proximal side wall and the second proximal side of the second mass, and a second distal spring disposed between the second distal side wall and the second distal side of the second mass;
a third housing member comprising a third proximal sidewall and a third distal sidewall, the third proximal sidewall and the third distal sidewall in opposite facing spaced-apart relation, a third mass disposed between the third proximal side wall and the third distal side wall, the third mass comprising a third proximal side and a third distal side, a third proximal spring disposed between the third proximal side wall and the third proximal side of the third mass, and a third distal spring disposed between the third distal side wall and the third distal side of the third mass;
a fourth housing member comprising a fourth proximal sidewall and a fourth distal sidewall, the fourth proximal sidewall and the fourth distal sidewall in opposite facing spaced-apart relation, a fourth mass disposed between the fourth proximal side wall and the fourth distal side wall, the fourth mass comprising a fourth proximal side and a fourth distal side, a fourth proximal spring disposed between the fourth proximal side wall and the fourth proximal side of the fourth mass, and a fourth distal spring disposed between the fourth distal side wall and the fourth distal side of the fourth mass;
wherein the first housing member is flexibly linked to the second housing member, the second housing member is flexibly linked to the third housing member, the third housing member is flexibly linked to the fourth housing member, and the fourth housing member is flexibly linked to the first housing member thereby forming an interconnected band adapted to encircle a limb of the user.

3. The tremor reduction device of claim 2 wherein the first proximal sidewall and the first distal sidewall are attached to a first base plate.

4. The tremor reduction device of claim 3 wherein the first base plate comprises a sliding means and the first mass slidably translates along the sliding means.

5. The tremor reduction device of claim 4 wherein the sliding means comprises a first rail member.

6. The tremor reduction device of claim 2 wherein the first mass translates along a first axis, the second mass translates along a second axis, the third mass translates along a third axis, and the fourth mass translates along a fourth axis.

7. The tremor reduction device of claim 6 wherein the first axis is in parallel alignment with the second axis and the third axis is in parallel alignment with the fourth axis.

8. The tremor reduction device of claim 7 wherein the first side member and the second side member reduce a tremor along a first translational direction and the third side member and the fourth side member reduce a tremor along a second translational direction.

9. The tremor reduction device of claim 7 wherein a tremor having a rotational direction is reduced by a collective interaction of the first side member, the second side member, the third side member, and the fourth side member.

10. A tremor reduction device worn by a user, the device comprising:
a first member comprising a first mass translatable along a first axis between a first proximal limit and a first distal limit, the first mass having a first neutral position midway between the first proximal limit and the first distal limit, where, following imposition of a first force having a component along the first axis, a first biasing means returns the first mass to the first neutral position;
a second member comprising a second mass translatable along a second axis between a second proximal limit and a second distal limit, the second mass having a second neutral position midway between the second proximal limit and the second distal limit, where, following following imposition of a second force having a component along the second axis, a second biasing means returns the second mass to the second neutral position;
a third member comprising a third mass translatable along a third axis between a third proximal limit and a third distal limit, the third mass having a third neutral position midway between the third proximal limit and the third distal limit, where, following imposition of a third force having a component along the third axis, a third biasing means returns the third mass to the third neutral position;
a fourth member comprising a fourth mass translatable along a fourth axis between a fourth proximal limit and a fourth distal limit, the fourth mass having a fourth neutral position midway between the fourth proximal limit and the fourth distal limit, where, following imposition of a fourth force having a component along the fourth axis, a fourth biasing means returns the fourth mass to the fourth neutral position;
wherein the first member is flexibly linked to the second member, the second member is flexibly linked to the third member, the third side member is flexibly linked to the fourth side member, and the fourth side member is flexibly linked to the first side member thereby forming an interconnected band adapted to encircle a limb of the user.

11. The tremor reduction device of claim 10 wherein the first axis is in parallel alignment with the second axis.

12. The tremor reduction device of claim 11 wherein a translation of the first mass along the first axis and a translation of the second mass along the second axis collectively reduce tremor along a first translational direction.

13. The tremor reduction device of claim 12 wherein the third axis is in parallel alignment with the fourth axis.

14. The tremor reduction device of claim 13 wherein a translation of the third mass along the third axis and a translation of the fourth mass along the fourth axis collectively reduce tremor along a second translational direction.

15. The tremor reduction device of claim 14 wherein a tremor having a rotational direction is reduced by, in sum, a first force caused by the translation of the first mass along the first axis, a second force caused by the translation of the second mass along the second axis, a third force caused by the translation of the third mass along the third axis, and a fourth force caused by the translation of the fourth mass along the fourth axis.

16. A method for reducing tremors in a limb of a person caused by Parkinson's disease or essential tremor, the method comprising:
measuring a horizontal frequency of the tremors;
measuring a vertical frequency of the tremors;
configuring a tremor reduction apparatus comprising a plurality of housings linked together in a closed loop configuration, each of the plurality of housings comprising a mass translatable along an axis between a proximal limit and a distal limit and having a neutral position there-between, each of the plurality of housings further comprising a biasing means for returning the mass to the neutral position, wherein the mass and biasing means are sized according to the horizontal frequency and the vertical frequency; and
disposing the tremor reduction apparatus about the limb of the person.

17. The method of claim 16 wherein the plurality of housings comprise a first housing member, a second housing member, a third housing member and a fourth housing member.

18. The method of claim 17 wherein the first housing member has a proximal sidewall and a distal sidewall, and the proximal sidewall defines the proximal limit and the distal sidewall defines the distal limit.

19. The method of claim 18 wherein the mass in the first housing member comprises a proximal side and a distal side.

20. The method of claim 19 wherein the biasing means comprises a proximal spring disposed between the proximal sidewall and the proximal side of the mass, the biasing means further comprising a distal spring disposed between the distal sidewall and the distal side of the mass.
